(19) United States
(12) Patent Application Publication
(10) Pub. No.: US 2026/0026902 A1
(43) Pub. Date: Jan. 29, 2026

(54) ROBOTIC SURGICAL SYSTEM WITH MULTIPLE PURPOSE SURGICAL CLIP APPLIER

(71) Applicant: Covidien LP, Mansfield, MA (US)

(72) Inventors: Henry E. Holsten, Sumter, SC (US); Matthew S. Hartzsch, Glastonbury, CT (US); Garrett P. Ebersole, Hamden, CT (US)

(21) Appl. No.: 18/994,161

(22) PCT Filed: Jul. 11, 2023
(86) PCT No.: PCT/IB2023/057080

§ 371 Date: Jan. 14, 2025

Related U.S. Application Data
(60) Provisional application No. 63/391,377, filed on Jul. 22, 2022.

(51) Int. Cl. A61B 34/37; A61B 17/00; A61B 17/128; A61B 17/29; A61B 34/00; A61B 34/30; A61B 90/00

(52) U.S. Cl. CPC A61B 34/37; A61B 17/1285; A61B 17/29; A61B 34/25; A61B 90/08; A61B 90/36; A61B 2017/00017; A61B 2017/2926; A61B 2034/305; A61B 2090/066; A61B 2090/0807

(57) ABSTRACT

A surgical robotic system includes a robotic arm having an instrument drive unit with at least one motor. The system also includes an instrument configured to couple to the instrument drive unit and to be actuated by the at least one motor. The instrument also includes an end effector having a pair of opposing jaws movable relative to each other. The end effector is further configured to engage a surgical attachment of a plurality of surgical attachments, which include a surgical clip, a grasper attachment, and a clip remover attachment. The system further includes a processor and a non-transitory computer readable medium storing a plurality of software controllers and instructions which. when executed by the processor, cause the processor to: identify a type of the surgical attachment engaged by the end effector and load a software controller of the stored plurality of software controllers based on the type of the surgical attachment engaged by the end effector. The software controller includes at least one parameter for controlling the instrument drive unit in a manner specific to the type of the surgical attachment engaged by the end effector.

### BACKGROUND

Surgical robotic systems generally include a surgeon console controlling one or more surgical robotic arms, each including a surgical instrument having an end effector (e.g., forceps or grasping instrument). In operation, the robotic arm is moved to a position over a patient and the surgical instrument is guided into a small incision via a surgical access port or a natural orifice of a patient to position the end effector at a work site within the patient's body. The surgeon console includes hand controllers which translate user input into movement of the surgical instrument and/or end effector.

Surgical robotic systems are configured to operate a variety of surgical instruments. However, exchanging instruments is a time-consuming process. Thus, there is a need for multi-purpose instruments that minimize instrument exchanges.

### SUMMARY

The present disclosure provides for a surgical robotic system having a robotic arm configured to couple to and actuate a multi-purpose clip applier tool. The clip applier tool is configured to apply clips as well as operate with a plurality of attachments, including a grasper tool and a clip removal tool.

According to one embodiment of the present disclosure, a surgical robotic system is disclosed. The surgical robotic system includes a robotic arm having an instrument drive unit with at least one motor. The system also includes an instrument configured to couple to the instrument drive unit and to be actuated by the at least one motor. The instrument also includes an end effector having a pair of opposing jaws movable relative to each other. The end effector is further configured to engage a surgical attachment of a plurality of surgical attachments, which include a surgical clip, a grasper attachment, and a clip remover attachment. The system further includes a processor and a non-transitory computer readable medium storing a plurality of software controllers and instructions which, when executed by the processor, cause the processor to: identify a type of the surgical attachment engaged by the end effector and load a software controller of the stored plurality of software controllers based on the type of the surgical attachment engaged by the end effector. The software controller includes at least one parameter for controlling the instrument drive unit in a manner specific to the type of the surgical attachment engaged by the end effector.

Implementations of the above embodiment may include one or more of the following features. According to one aspect of the above embodiment, the instrument drive unit may further include a torque sensor configured to measure a torque of the at least one motor during engagement of the end effector with the surgical attachment engaged by the end effector. The instructions when executed by the processor may further cause the processor to identify the type of the surgical attachment engaged by the end effector based on the torque. The surgical robotic system may also include a user interface configured to receive a user input indicating the type of the surgical attachment engaged by the end effector. The instructions when executed by the processor may further cause the processor to identify the type of the surgical attachment engaged by the end effector based on the user input. The surgical robotic system may include a surgeon console that may include at least one handle controller configured to control the instrument and a control tower coupled to the surgeon console and the robotic arm. The user interface may be disposed on at least one of the surgeon console or the control tower. The user interface may be displayed on a touch screen.

According to another embodiment of the present disclosure, a powered surgical instrument is disclosed. The powered surgical instrument includes an end effector having a pair of opposing jaws movable relative to each other and configured to engage a surgical attachment of a plurality of surgical attachments, which include a surgical clip, a grasper attachment, and a clip remover attachment. The instrument also includes a motor configured to actuate the end effector. The instrument further includes a processor and a non-transitory computer readable medium storing a plurality of software controllers and instructions which, when executed by the processor, cause the processor to identify a type of the surgical attachment and load a software controller of the stored plurality of software controllers based on the type of the surgical attachment engaged by the end effector. The software controller includes at least one parameter for controlling the motor in a manner specific to the type of the surgical attachment engaged by the end effector.

Implementations of the above embodiment may include one or more of the following features. According to one aspect of the above embodiment, the powered surgical instrument may include a torque sensor configured to measure a torque of the motor during engagement of the end effector with the surgical attachment engaged by the end effector. The instructions when executed by the processor may further cause the processor to identify the type of the surgical attachment engaged by the end effector based on the torque. The powered surgical instrument may also include a user interface configured to receive a user input indicating the type of the surgical attachment engaged by the end effector. The instructions when executed by the processor may further cause the processor to identify the type of the surgical attachment engaged by the end effector based on the user input. Each of the jaws may include a surface feature configured to engage a boss disposed on each surgical attachment of the plurality of surgical attachments. The plurality of surgical attachments may include a grasper attachment having a pair of opposing plates interconnected by a biasing member, each opposing plate may include the boss and be configured to engage one jaw of the pair of opposing jaws. The plurality of surgical attachments may include a clip remover attachment having a pair of opposing decoupled plates, each opposing plate may include the boss and be configured to engage one jaw of the pair of opposing jaws.

According to a further embodiment of the present disclosure, a method for controlling a powered surgical instrument is disclosed. The method includes receiving an indication of engagement of a surgical attachment of a plurality of surgical attachments by an end effector including a pair of opposing jaws movable relative to each other, the plurality of surgical attachments including a surgical clip, a grasper attachment, and a clip remover. The end effector includes a pair of opposing jaws movable relative to each other. The method also includes identifying, at a processor, a type of the surgical attachment engaged by the end effector. The method further includes loading a software controller, at the processor, based on the type of the surgical attachment engaged by the end effector. The software controller includes at least one parameter for controlling a motor configured to actuate the end effector in a manner specific to the type of the surgical attachment engaged by the end effector.

Implementations of the above embodiment may include one or more of the following features. According to one aspect of the above embodiment, the method may also include measuring torque of the motor during coupling of the end effector with the surgical attachment engaged by the end effector and identifying, at the processor, the type of the surgical attachment engaged by the end effector based on the measured torque. The method may further include receiving a user input at a user interface indicating the type of the surgical attachment engaged by the end effector and identifying, at the processor, the type of the surgical attachment engaged by the end effector based on the user input. The method may also include processing, at the processor, an image of the end effector and the surgical attachment engaged by the end effector and identifying, at the processor, the type of the surgical attachment engaged by the end effector based on the processed image.

### BRIEF DESCRIPTION OF THE DRAWINGS

Various embodiments of the present disclosure are described herein with reference to the drawings wherein:

FIG. 1 is a schematic illustration of a surgical robotic system including a control tower, a console, and one or more surgical robotic arms each disposed on a mobile cart according to an embodiment of the present disclosure;

FIG. 2 is a perspective view of a surgical robotic arm of the surgical robotic system of FIG. 1 according to an embodiment of the present disclosure;

FIG. 3 is a perspective view of a mobile cart having a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure;

FIG. 5 is a perspective view, with parts separated, of an instrument drive unit and a surgical instrument according to an embodiment of the present disclosure;

FIG. 6 is a side view of a surgical clip;

FIG. 7 is a perspective view of an end effector of a multi-purpose clip applier with a grasper attachment according to an embodiment of the present disclosure;

FIG. 8 is a perspective view of a cartridge for storing surgical attachments including surgical clips, grasper attachments, and a clip remover attachment according to an embodiment of the present disclosure;

FIG. 9 is a perspective view of the grasper attachment of FIG. 7;

FIG. 10 is a perspective view of a grasper attachment according to another embodiment of the present disclosure;

FIG. 11 is a perspective view of the end effector of the multi-purpose clip applier with the grasper attachment of FIG. 10;

FIG. 12 is a perspective view of the end effector of the multi-purpose clip applier with a clip remover according to an embodiment of the present disclosure;

FIG. 13 is a perspective view of the clip remover attachment of FIG. 12; and

FIG. 14 is a flow chart of a method for determining a surgical attachment coupled to the clip applier and controlling the clip applier according to an embodiment of the present disclosure.

### DETAILED DESCRIPTION

Embodiments of the presently disclosed surgical robotic system are described in detail with reference to the drawings, in which like reference numerals designate identical or corresponding elements in each of the several views.

As will be described in detail below, the present disclosure is directed to a surgical robotic system, which includes a surgeon console, a control tower, and one or more mobile carts having a surgical robotic arm coupled to a setup arm. The surgeon console receives user input through one or more interface devices, which are processed by the control tower as movement commands for moving the surgical robotic arm and an instrument and/or camera coupled thereto. Thus, the surgeon console enables teleoperation of the surgical arms and attached instruments/camera. The surgical robotic arm includes a controller, which is configured to process the movement command and to generate a torque command for activating one or more actuators of the robotic arm, which would, in turn, move the robotic arm in response to the movement command.

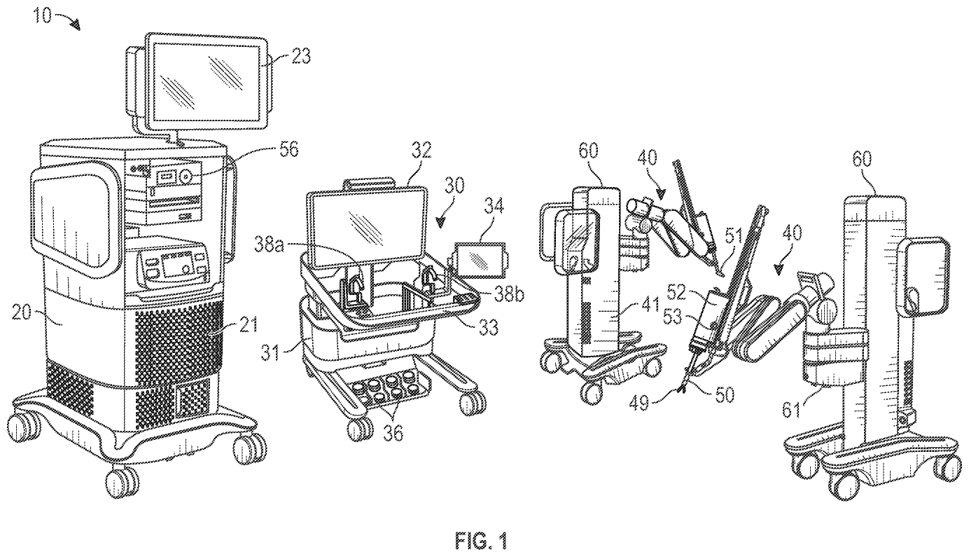

With reference to FIG. 1, a surgical robotic system 10 includes a control tower 20, which is connected to all of the components of the surgical robotic system 10 including a surgeon console 30 and one or more movable carts 60. Each of the movable carts 60 includes a robotic arm 40 having a surgical instrument 50 removably coupled thereto. The robotic arms 40 also couple to the movable carts 60. The robotic system 10 may include any number of movable carts 60 and/or robotic arms 40.

The surgical instrument 50 is configured for use during minimally invasive surgical procedures. In embodiments, the surgical instrument 50 may be configured for open surgical procedures. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue. In yet further embodiments, the surgical instrument 50 may be a surgical clip applier including a pair of jaws configured apply a surgical clip onto tissue.

One of the robotic arms 40 may include an endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to a video processing device 56, which may be disposed within the control tower 20. The video processing device 56 may be any computing device as described below configured to receive the video feed from the endoscopic camera 51 and output the processed video stream.

The surgeon console 30 includes a first display 32, which displays a video feed of the surgical site provided by camera 51 of the surgical instrument 50 disposed on the robotic arm 40, and a second display 34, which displays a user interface for controlling the surgical robotic system 10. The first display 32 and second display 34 may be touchscreens allowing for displaying various graphical user inputs.

The surgeon console 30 also includes a plurality of user interface devices, such as foot pedals 36 and a pair of handle controllers 38a and 38b which are used by a user to remotely control robotic arms 40. The surgeon console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgeon console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgeon console 30, in such a way that robotic arms 40 and the surgical instrument 50 execute a desired movement sequence in response to input from the foot pedals 36 and the handle controllers 38a and 38b. The foot pedals 36 may be used to enable and lock the hand controllers 38a and 38b, repositioning camera movement and electrosurgical activation/deactivation. In particular, the foot pedals 36 may be used to perform a clutching action on the hand controllers 38a and 38b. Clutching is initiated by pressing one of the foot pedals 36, which disconnects (i.e., prevents movement inputs) the hand controllers 38a and/or 38b from the robotic arm 40 and corresponding instrument 50 or camera 51 attached thereto. This allows the user to reposition the hand controllers 38a and 38b without moving the robotic arm(s) 40 and the instrument 50 and/or camera 51. This is useful when reaching control boundaries of the surgical space.

Each of the control tower 20, the surgeon console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-1203 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

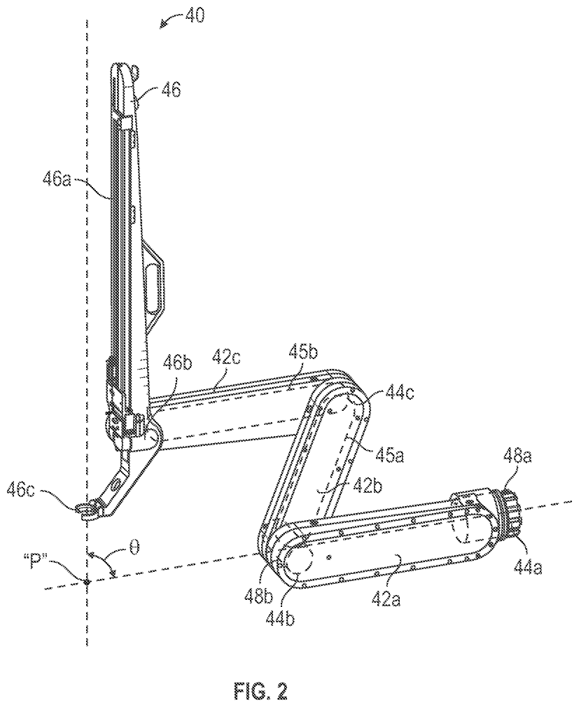

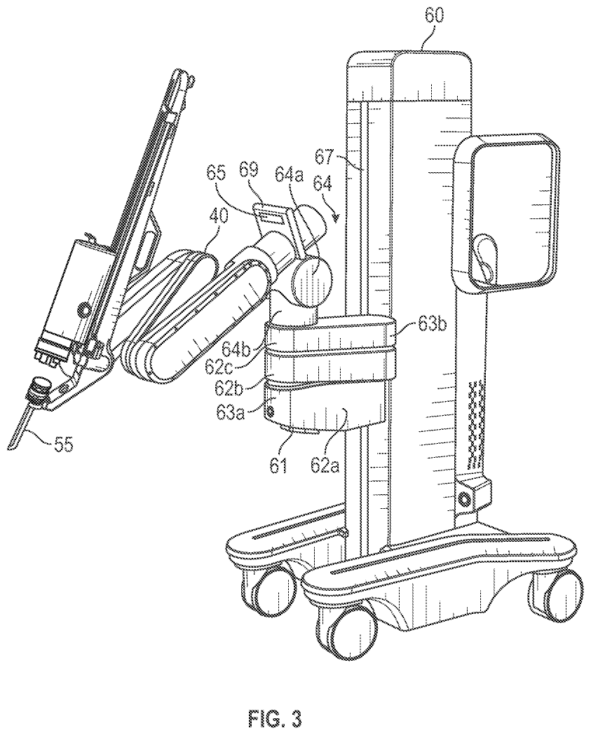

With reference to FIG. 2, each of the robotic arms 40 may include a plurality of links 42a, 42b, 42c, which are interconnected at joints 44a, 44b, 44c, respectively. Other configurations of links and joints may be utilized as known by those skilled in the art. The joint 44a is configured to secure the robotic arm 40 to the mobile cart 60 and defines a first longitudinal axis. With reference to FIG. 3, the mobile cart 60 includes a lift 67 and a setup arm 61, which provides a base for mounting of the robotic arm 40. The lift 67 allows for vertical movement of the setup arm 61. The mobile cart 60 also includes a display 69 for displaying information pertaining to the robotic arm 40. In embodiments, the robotic arm 40 may include any type and/or number of joints.

The setup arm 61 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 61 includes controls 65 for adjusting movement of the links 62a, 62b, 62c as well as the lift 67. In embodiments, the setup arm 61 may include any type and/or number of joints.

The third link 62c may include a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and a holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. In other words, the pivot point “P” is a remote center of motion (RCM) for the robotic arm 40. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the holder 46 defines a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components an end effector 49 of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic access port 55 (FIG. 3) held by the holder 46. The holder 46 also includes a port latch 46c for securing the access port 55 to the holder 46 (FIG. 2).

The robotic arm 40 also includes a plurality of manual override buttons 53 (FIG. 1) disposed on the IDU 52 and the setup arm 61, which may be used in a manual mode. The user may press one or more of the buttons 53 to move the component associated with the button 53.

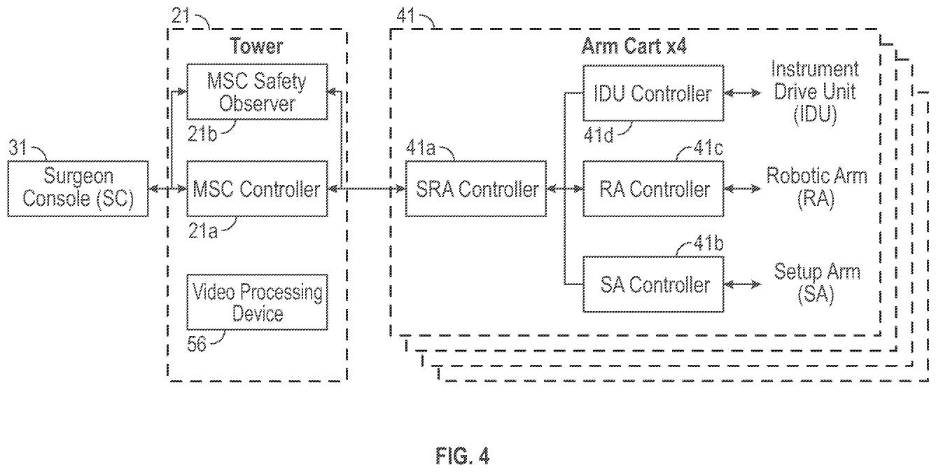

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgeon console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgeon console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the mobile cart 60, the robotic arm 40, and the IDU 52. The main cart controller 41a also communicates actual joint angles back to the controller 21a.

Each of joints 63a and 63b and the rotatable base 64 of the setup arm 61 are passive joints (i.e., no actuators are present therein) allowing for manual adjustment thereof by a user. The joints 63a and 63b and the rotatable base 64 include brakes that are disengaged by the user to configure the setup arm 61. The setup arm controller 41b monitors slippage of each of joints 63a and 63b and the rotatable base 64 of the setup arm 61, when brakes are engaged or can be freely moved by the operator when brakes are disengaged, but do not impact controls of other joints. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the handle controller controlling the robotic arm 40, e.g., the handle controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controllers 38a may be embodied as a coordinate position and roll-pitch-yaw (RPY) orientation relative to a coordinate reference frame, which is fixed to the surgeon console 30. The desired pose of the instrument 50 is relative to a fixed frame on the robotic arm 40. The pose of the handle controller 38a is then scaled by a scaling function executed by the controller 21a. In embodiments, the coordinate position may be scaled down and the orientation may be scaled up by the scaling function. In addition, the controller 21a may also execute a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

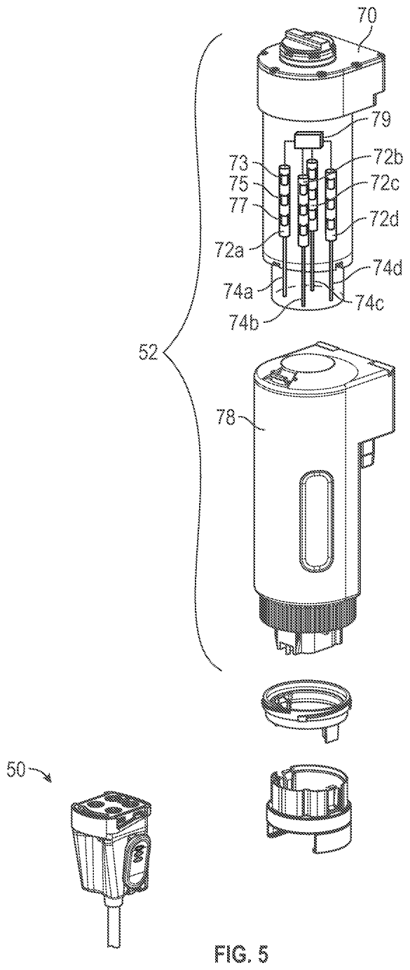

With reference to FIG. 5, the IDU 52 is shown in more detail and is configured to transfer power and actuation forces from its motors 72a, 72b, 72c, 72d to the instrument 50 to drive movement of components of the instrument 50, such as articulation, rotation, pitch, yaw, clamping, cutting, etc. The IDU 52 may also be configured for the activation or firing of an electrosurgical energy-based instrument or the like (e.g., cable drives, pulleys, friction wheels, rack and pinion arrangements, etc.).

The IDU 52 includes a motor pack 70 and a sterile barrier housing 78. Motor pack 70 includes motors 72a, 72b, 72c, 72d for controlling various operations of the instrument 50. The instrument 50 is removably couplable to IDU 52. As the motors 72a, 72b, 72c, 72d of the motor pack 70 are actuated, rotation of the drive transfer shafts 74a, 74b, 74c, 74d of the motors 72a, 72b, 72c, 72d, respectively, is transferred to the drive assemblies of the instrument 50. The instrument 50 is configured to transfer rotational forces/movement supplied by the IDU 52 (e.g., via the motors 72a, 72b, 72c, 72d of the motor pack 70) into longitudinal movement or translation of the cables or drive shafts to effect various functions of the end effector 49.

Each of the motors 72a, 72b, 72c, 72d includes a current sensor 73, a torque sensor 75, and an encoder sensor 77. For conciseness only operation of the motor 72a is described below. The sensors 73, 75, 77 monitor the performance of the motor 72a. The current sensor 73 is configured to measure the current draw of the motor 72a and the torque sensor 75 is configured to measure motor torque. The torque sensor 75 may be any force or strain sensor including one or more strain gauges configured to convert mechanical forces and/or strain into a sensor signal indicative of the torque output by the motor 72a. The encoder sensor 77 may be any device that provides a sensor signal indicative of the number of rotations of the motor 72a, such as a mechanical encoder or an optical encoder. Parameters which are measured and/or determined by the encoder sensor 77 may include speed, distance, revolutions per minute, position, and the like. The sensor signals from sensors 73, 75, 77 are transmitted to the IDU controller 41d, which then controls the motors 72a, 72b, 72c, 72d based on the sensor signals. In particular, the motors 72a, 72b, 72c, 72d are controlled by an actuator controller 79, which controls torque outputted and angular velocity of the motors 72a, 72b, 72c, 72d. In embodiments, additional position sensors may also be used, which include, but are not limited to, potentiometers coupled to movable components and configured to detect travel distances, Hall Effect sensors, accelerometers, and gyroscopes. In embodiments, a single controller can perform the functionality of the IDU controller 41d and the actuator controller 79. In embodiments, the instrument 50 may be part of a powered surgical instrument and may be coupled to a handheld surgical system having one or more motors similar to the IDU 52.

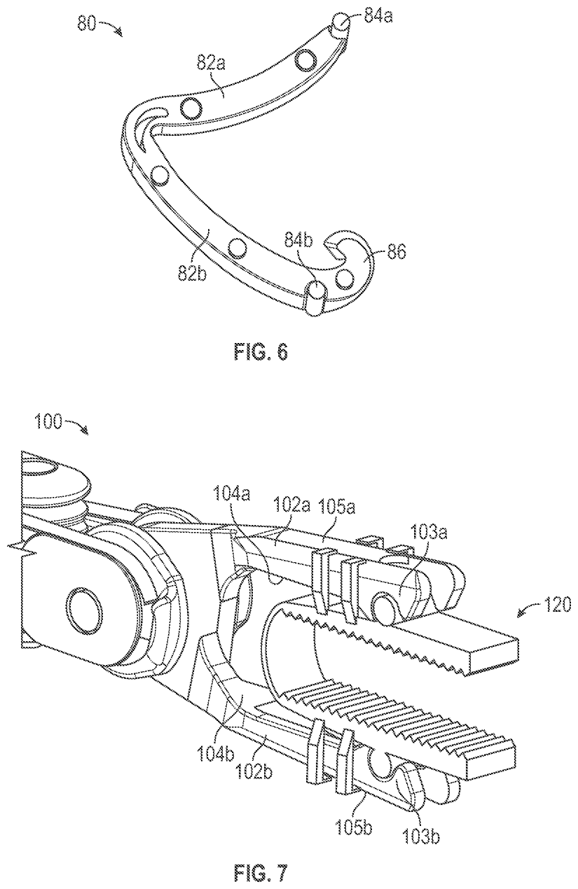

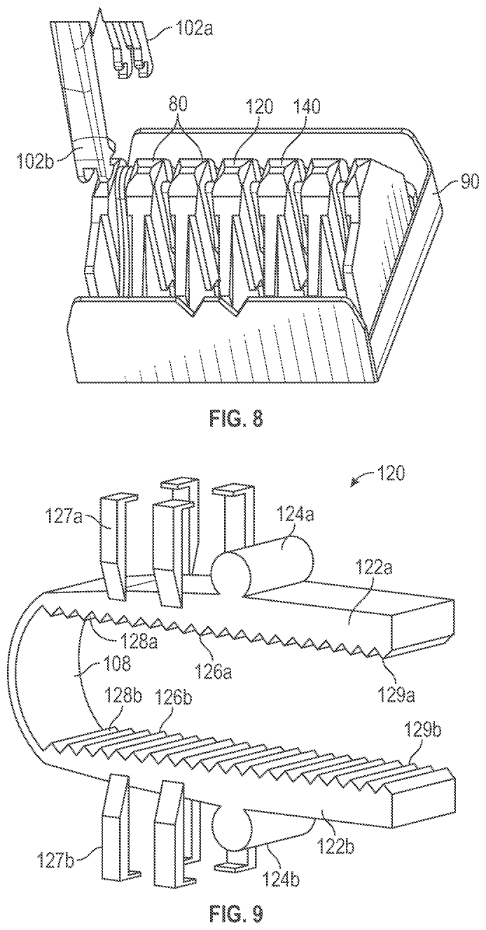

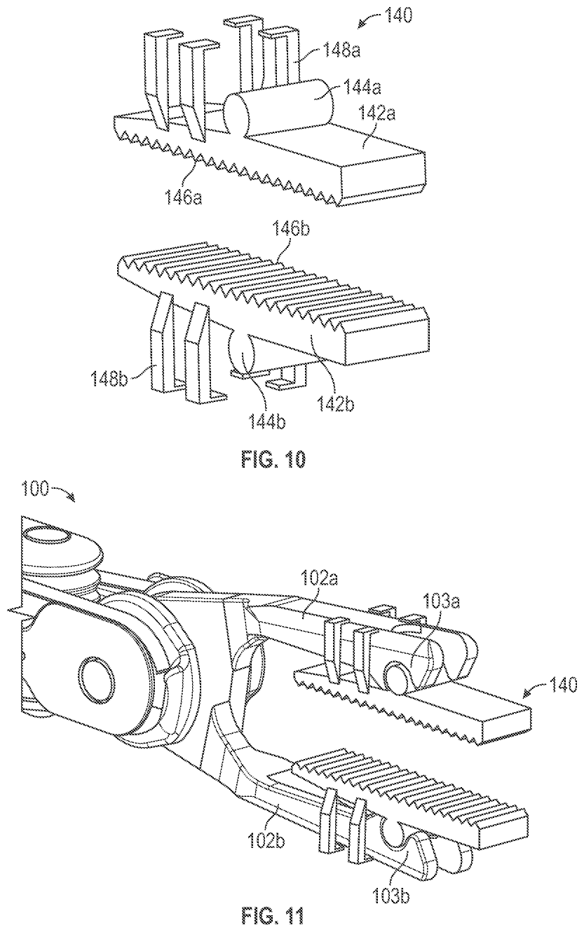

With reference to FIGS. 6 and 7, a clip applier 100 (i.e., end effector 49) is configured to engage different types of surgical attachments, such as a surgical clip 80 (FIG. 6), a grasper attachment 120 (FIG. 9), a grasper attachment 140 (FIG. 10), and a clip remover attachment 160 (FIG. 13). The clip applier 100 includes a pair of opposing jaws 102a and 102b is configured to engage the surgical clip 80, which may be used in ligating vessels. The clip 80 may be delivered inside the patient through the access port 55 and may be disposed on a cartridge 90 having a plurality of clips 80 enabling the clip applier 100 to be reloaded in situ as shown in FIG. 8.

The surgical clip 80 includes a pair of bendable arms 82a and 82b, each having a boss 84a and 84b, which may have a cylindrical shape. The bosses 84a and 84b are configured to engage corresponding surface features (e.g., concave cutouts) 103a and 103b disposed on inner portions 104a and 104b of the jaws 102a and 102b, respectively. Engagement of the bosses 84a and 84b with the surface features 103a and 103b retains the clip 80 insides the clip applier 100 until the clip is clamped and the arms 82a and 82b are locked to each other via a locking mechanism 86 by closing the jaws 102a and 102b. After the clip 80 is locked, the jaws 102a and 102b are opened and the clip 80 remains attached to the tissue.

With reference to FIG. 9, the clip applier 100 is also configured to operate with the grasper attachment 120, which enables the clip applier 100 to grasp and manipulate tissue. The grasper attachment 120 may be formed from stainless steel, or any other suitable resilient material. The grasper attachment 120 includes a pair of opposing plates 122a and 122b, each having a boss 124a and 124b, which may have a cylindrical shape. The bosses 124a an 124b are configured to engage corresponding surface features 103a and 103b of the jaws 102a and 102b, respectively. Each of the opposing plates 122a and 122b includes a tissue grasping surface 126a and 126b, respectively, which may be textured. The opposing plates 122a and 122b also include one or more hooked arms 127a and 127b configured to engage outer portions 105a and 105b of the jaws 102a and 102b. The arms 127a and 127b secure the opposing plates 122a and 122b, preventing separation from the jaws 102a and 102b, e.g., due to sticking to or being trapped in tissue.

The opposing plates 122a and 122b are interconnected at their proximal end portions 128a and 128b via a biasing member 108, which may be a curved leaf spring. The biasing member 108 pushes the opposing plates 122a and 122b apart as well as the jaws 102a and 102b. In addition, the biasing member 108 enables for distal end portions 129a and 129b of the opposing plates 122a and 122b to close at an acute angle relative to each other, i.e., non-parallel closure, which provides for better gripping. The biasing force is overcome by the IDU 52 when closing the jaws 102a and 102b to maintain a grip on the tissue grasped between the opposing plates 122a and 122b.

With reference to FIG. 10, the clip applier 100 is further configured to operate with the grasper attachment 140, which enables the clip applier 100 to grasp and manipulate tissue. The grasper attachment 140 may be formed from stainless steel, or any other suitable resilient material. The grasper attachment 140 includes a pair of opposing plates 142a and 142b, each having a boss 144a and 144b, which may have a cylindrical shape. The bosses 144a an 144b are configured to engage corresponding surface features 103a and 103b of the jaws 102a and 102b, respectively. Each of the opposing plates 142a and 142b includes a tissue grasping surface 146a and 146b, respectively, which may be fully or partially textured. The opposing plates 142a and 142b also include one or more hooked arms 148a and 148b configured to engage outer portions 105a and 105b of the jaws 102a and 102b. The arms 148a and 148b secure the opposing plates 142a and 142b, preventing separation from the jaws 102a and 102b, e.g., due to sticking to or being trapped in tissue.

The opposing plates 142a and 142b are decoupled from each other, e.g., do not include the biasing member 108 like the grasper attachment 120. This allows for the opposing plates 142a and 142b to close in a parallel manner as the opposing plates 142a and 142b pivot about the bosses 144a and 144b as the jaws 102a and 102b are closed.

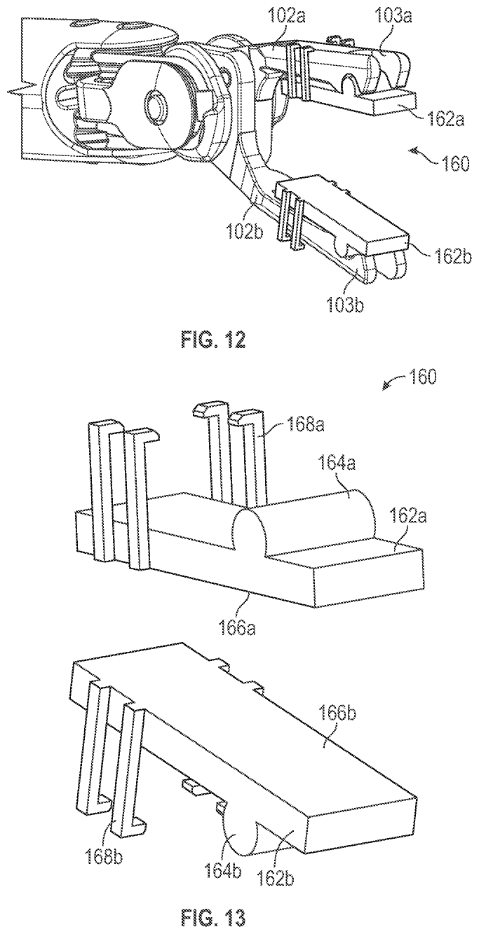

With reference to FIGS. 12 and 13, the clip applier 100 is further configured to operate with the clip remover attachment 160, which enables the clip applier 100 to remove the surgical clip 80. The clip remover attachment 160 may be formed from stainless steel, or any other suitable resilient material. The clip remover attachment 160 includes a pair of opposing plates 162a and 162b, each having a boss 164a and 164b, which may have a cylindrical shape. The bosses 164a an 164b are configured to engage corresponding surface features 103a and 103b of the jaws 102a and 102b, respectively. Each of the opposing plates 162a and 162b includes a clip grasping surface 166a and 166b, respectively, which may be flat or partially textured. The opposing plates 162a and 162b also include one or more hooked arms 168a and 168b configured to engage outer portions 105a and 105b of the jaws 102a and 102b. The arms 168a and 168b secure the opposing plates 162a and 162b, preventing separation from the jaws 102a and 102b, e.g., due to sticking to or being trapped in tissue.

The opposing plates 162a and 162b are decoupled from each other, e.g., do not include the biasing member 108 like the grasper attachment 120. This allows for the opposing plates 162a and 162b to close in a parallel manner as the opposing plates 162a and 162b pivot about the bosses 164a and 164b as the jaws 102a and 102b are closed. Parallel closure enables removal of the surgical clip 80. Clip removal includes grasping a closed surgical clip 80 between the opposing plates 162a and 162b and compressing the surgical clip 80 until the locking mechanism 86 is opened. Thereafter, the jaws 102a and 102b are opened, which allows the bendable arms 82a and 82b to open along with the jaws 102a and 102b, while the surgical clip 80 is maintained between the opposing plates 162a and 162b.

The grasper attachment 120 and the clip remover attachment 160 may be stored in the cartridge 90 along with the clips 80, as shown in FIG. 8. The clip applier 100 is configured to couple to a selected one of the surgical attachments (i.e., the surgical clip 80, the grasper attachment 120, or the clip remover attachment 160) at any given time. After using the grasper attachment 120 or the clip remover attachment 160, the clip applier 100 my return the surgical attachments to the cartridge 90.

The grasper attachments 120 and 140 and the clip remover attachment 160 may be stored in the cartridge 90 along with the clips 80, as shown in FIG. 8. The clip applier 100 is configured to couple to a selected one of the surgical attachments (i.e., the surgical clip 80, the grasper attachments 120 and/or 140, the clip remover attachment 160) at any given time. After using the grasper attachments 120 and/or 140 or the clip remover attachment 160, the clip applier 100 my return the surgical attachments to the cartridge 90.

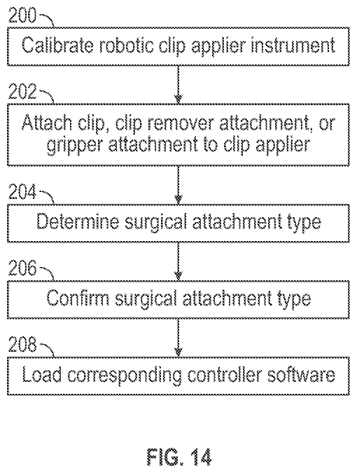

With reference to FIG. 14, a method for determining the type of the surgical attachment coupled to the clip applier 100 includes calibrating the clip applier 100 at step 200. Calibration of the clip applier 100 is performed after coupling the clip applier 100 to the IDU 52 of the robotic arm 40. Calibration is performed for each of the degrees of freedom, e.g., yaw, pitch, and jaw angle between jaws 102a and 102b. Calibration may be accomplished by moving the clip applier 100 about each of the axes “A-A” and “B-B” as well as opening the jaws 102a and 102b between two limits of corresponding motion range. Calibration may be performed within an access port 55, which provides mechanical limits for calibrating the clip applier 100. In particular, moving the clip applier 100 and/or the jaws 102a and 102b during calibration until contacting the inner surfaces of the access port 55, which are used as mechanical limits, i.e., end stops. During yaw calibration, the clip applier 100 is pivoted in either direction until the clip applier 100 reaches the end stop by hitting the walls of the access port 55. Reaching the end stop may be detected by the torque sensors 75 of the motors 72a-d. Pitch calibration is performed similarly to yaw calibration, except that the clip applier 100 is pivoted about a pivot axis. Jaw angle calibration is performed by moving, i.e., opening, both jaws 102a and 102b until each of the jaws 102a and 102b hits the walls of the access port 55.

After calibration, the clip applier 100 is inserted fully through the access port 55 and is maneuvered to pick up a surgical attachment from the cartridge 90 at step 202. The user moves the clip applier 100 to the cartridge 90, which may be disposed on another access port 55. The user then opens the jaws 102a and 102b over the surgical attachment and closes the jaws 102a and 102b to engage the surgical attachment engaged by the end effector.

The system 10 automatically determines the type of the surgical attachment that the clip applier 100 grabbed in step 202. One or more processors of the system 10, may be configured to execute instructions stored in memory, which are configured to cause the processor, e.g., controller 21a, to determine the type of the surgical attachment selected. The controller 21a is configured to determine the type of the surgical attachment based on image processing (e.g., image recognition) of video feed from the camera 51 by the video processing device 56, and/or position and torque measurements, and/or user input, e.g., through a graphical user interface (GUI), which may be used to confirm surgical attachment type.

At step 204, the controller 21a determines the type of the surgical attachment based on image processing and/or sensor data from the sensors 73, 75, 77 and/or calibration data. With respect to image processing, the video feed from the camera 51 may be analyzed using any suitable image recognition techniques to determine the type of the surgical attachment coupled to the clip applier 100. The controller 21a may use a machine learning algorithm trained on an image data set of surgical attachments, allowing the controller 21a to identify the surgical attachment by analyzing the images from the camera 51 using the machine learning algorithm.

Alternatively, or additionally to image recognition, sensor data from the sensors 73, 75, 77 may also be used to determine the type of the surgical attachment. During engagement of the clip applier 100 with the surgical attachment, torque or other motor parameters are measured and provided to the controller 21a, which compares the torque recorded during coupling of the clip applier 100 to the surgical attachment to different thresholds since engagement of the clip applier 100 with different surgical attachment results in different torque levels.

Image processing determination by the controller 21a may be cross-referenced with the determination based on the sensor feedback and output a confidence value to the user. At step 206, the user may confirm or input the type of the surgical attachment. The GUI may be touch-enabled and may include one or more windows, menus, icons, tabs, a slider, or any other suitable selection interface. The GUI may be displayed on one of the displays 23, 32, 34 listing a plurality of surgical attachments, allowing the surgeon to select the surgical attachment type.

Once the surgical attachment is identified, the controller 21a loads a software controller corresponding to the surgical attachment engaged by the end effector at step 208. The software controller 21a includes instrument control parameters specific to the type of the surgical attachment including, torque thresholds, which may be different for each of the surgical clip 80, the grasper attachment 120, the grasper attachment 140, and the clip remover attachment 160.

In addition, the software controller 21a may include control algorithms for each of the surgical attachments. During the surgical procedure, the user may exchange multiple surgical attachments, and once a new surgical attachment is selected, the system 10 proceeds through the method of FIG. 12.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

What is claimed is:

1. A surgical robotic system comprising:
a robotic arm including an instrument drive unit having at least one motor;
an instrument configured to couple to the instrument drive unit and to be actuated by the at least one motor, the instrument including an end effector including a pair of opposing jaws movable relative to each other, the end effector configured to engage a surgical attachment of a plurality of surgical attachments including a surgical clip, a grasper attachment, and a clip remover attachment;
a processor; and
a non-transitory computer readable medium storing instructions which, when executed by the processor, cause the processor to:
identify a type of the surgical attachment engaged by the end effector; and
load a software controller based on the type of the surgical attachment engaged by the end effector, the software controller including at least one parameter for controlling the instrument drive unit in a manner specific to the type of the surgical attachment engaged by the end effector.

2. The surgical robotic system according to claim 1, wherein the instrument drive unit further includes a torque sensor configured to measure a torque of the at least one motor during engagement of the end effector with the surgical attachment engaged by the end effector.

3. The surgical robotic system according to claim 2, wherein the instructions when executed by the processor further cause the processor to identify the type of the surgical attachment engaged by the end effector based on the torque.

4. The surgical robotic system according to claim 1, further comprising:
a user interface configured to receive a user input indicating the type of the surgical attachment engaged by the end effector.

5. The surgical robotic system according to claim 4, wherein the instructions when executed by the processor further cause the processor to identify the type of the surgical attachment engaged by the end effector based on the user input.

6. The surgical robotic system according to claim 4, further comprising:
a surgeon console including at least one handle controller configured to control the instrument; and
a control tower coupled to the surgeon console and the robotic arm.

7. The surgical robotic system according to claim 6, wherein the user interface is disposed on at least one of the surgeon console or the control tower.

8. The surgical robotic system according to claim 7, wherein the user interface is displayed on a touch screen.

9. A powered surgical instrument comprising:
an end effector including a pair of opposing jaws movable relative to each other and configured to engage a surgical attachment of a plurality of surgical attachments including a surgical clip, a grasper attachment, and a clip remover attachment;
a motor configured to actuate the end effector;
a processor; and
a non-transitory computer readable medium storing instructions which, when executed by the processor, cause the processor to:
identify a type of the surgical attachment engaged by the end effector; and
load a software controller based on the type of the surgical attachment engaged by the end effector, the software controller including at least one parameter for controlling the motor in a manner specific to the type of the surgical attachment engaged by the end effector.

10. The powered surgical instrument according to claim 9, further comprising:
a torque sensor configured to measure a torque of the motor during engagement of the end effector with the surgical attachment engaged by the end effector.

11. The powered surgical instrument according to claim 10, wherein the instructions when executed by the processor further cause the processor to identify the type of the surgical attachment engaged by the end effector based on the torque.

12. The powered surgical instrument according to claim 9, further comprising:
a user interface configured to receive a user input indicating the type of the surgical attachment engaged by the end effector.

13. The powered surgical instrument according to claim 12, wherein the instructions when executed by the processor further cause the processor to identify the type of the surgical attachment engaged by the end effector based on the user input.

14. The powered surgical instrument according to claim 9, wherein each of the jaws includes a surface feature configured to engage a boss disposed on each surgical attachment of the plurality of surgical attachments.

15. The powered surgical instrument according to claim 14, wherein the surgical attachment engaged by the end effector is the grasper attachment which includes a pair of opposing plates interconnected by a biasing member, each opposing plate including the boss and being configured to engage one jaw of the pair of opposing jaws.

16. The powered surgical instrument according to claim 14, wherein the surgical attachment engaged by the end effector is the clip remover attachment which includes a pair of opposing decoupled plates, each opposing plate including the boss and being configured to engage one jaw of the pair of opposing jaws.

17. A method for controlling a powered surgical instrument, the method comprising:
receiving an indication of engagement of a surgical attachment of a plurality of surgical attachments by an end effector including a pair of opposing jaws movable relative to each other, the plurality of surgical attachments including a surgical clip, a grasper attachment, and a clip remover attachment;
identifying, at a processor, a type of the surgical attachment engaged by the end effector; and
loading a software controller, at the processor, based on the type of the surgical attachment, the software controller including at least one parameter for controlling a motor configured to actuate the end effector in a manner specific to the type of the surgical attachment.

18. The method according to claim 17, further comprising:
measuring torque of the motor during coupling of the end effector with the surgical attachment; and
identifying, at the processor, the type of the surgical attachment based on the measured torque.

19. The method according to claim 17, further comprising:
receiving a user input at a user interface indicating the type of the surgical attachment; and
identifying, at the processor, the type of the surgical attachment based on the user input.

20. The method according to claim 17, further comprising:
processing, at the processor, an image of the end effector and the surgical attachment; and
identifying, at the processor, the type of the surgical attachment based on the processed image.
